(19) United States
(12) Patent Application Publication
(10) Pub. No.: US 2016/0235441 A1
(43) Pub. Date: Aug. 18, 2016

(54) APPARATUS FOR EXTRACTING HAIR FOLLICLES FOR USE IN TRANSPLANTATION

(71) Applicant: Roger Parkin, Garnet Valley, PA (US)

(72) Inventor: Roger Parkin, Garnet Valley, PA (US)

(21) Appl. No.: 15/135,755

(22) Filed: Apr. 22, 2016

Related U.S. Application Data
(63) Continuation-in-part of application No. PCT/US2014/061747, filed on Oct. 22, 2014.
(60) Provisional application No. 61/894,056, filed on Oct. 22, 2013.

(51) Int. Cl. A61B 17/34; A61B 17/3205

(52) U.S. Cl. CPC A61B 17/3468; A61B 17/32053; A61B 2017/00969

(57) ABSTRACT

An apparatus for surgical extraction of hair follicles for transplantation uses a rotating needle for cutting follicular units from a patient's scalp. Each follicular unit is drawn through the needle and transported through a diverter unit to a selected one of plural collectors, in which the follicular units are accumulated for transplantation. The rotating needle is extends through a seal compartment in which a seal, composed of a spring inside a spring carrier, engages both the needle and a cylindrical inner wall of the seal compartment.

### CROSS-REFERENCE TO RELATED APPLICATIONS

This application is a continuation-in-part of International Application PCT/US2014/61747, filed Oct. 22, 2014, and claims priority from U.S. Provisional Patent Application 61/894,056, filed Oct. 22, 2013.

### FIELD OF THE INVENTION

This invention relates to the field of surgical hair restoration, and particularly to apparatus for follicular unit extraction.

### BACKGROUND OF THE INVENTION

Follicular unit transplantation is one of several known procedures for surgical treatment of baldness. In this procedure, small plugs, each comprising a hair follicle, are surgically removed from an area of the patient's scalp on which hair is actively growing, and implanted into a balding area. Extraction is usually carried out by using a hollow punch to make a circular incision around a follicular unit and remove the follicular unit from the scalp. Suction can be used to draw the follicular unit through the punch to a collection device from which it can be removed for implantation.

Examples of instruments for follicular unit extraction using suction are described in U.S. Pat. No. 5,827,297, granted Oct. 27, 1998, and U.S. Pat. No. 6,572,625, granted Jun. 3, 2003.

U.S. Pat. No. 5,827,297 describes a device for hair transplantation in which extraction and transplantation are integrated into a single instrument in which a follicular unit is extracted and immediately implanted before a next follicular unit is extracted. The instrument in U.S. Pat. No. 5,827,297 uses a motor-driven rotating punch, connected to a fixed conduit by a seal.

The apparatus described in U.S. Pat. No. 6,572,625 has the capability of extracting and collecting plural follicular units for subsequent reimplantation. There are other instruments currently in use that collect plural follicular units prior to implantation.

Especially when multiple units are collected for subsequent reimplantation, it is important to take measures to preserve the extracted follicles, as they remain viable only for a very limited time.

A follicular can comprise a single hair, two hairs, or three or more hairs. For esthetically pleasing results it is ordinarily desirable to reimplant single hair units adjacent the hair line, to reimplant the units comprising two hairs farther from the hair line, and to reimplant the units comprising three or more hairs still farther from the hairline. If plural follicular units are collected prior to implantation, the different kinds of follicular units, i.e., single hair units, two hair units, and units having three or more hairs, become mixed with one another in the collection device, requiring sorting, which is time-consuming, and can result in loss of viability.

A problem encountered in the use of a motor-driven rotating punch is that blood, tissue and other materials that accompany the follicular unit as it is extracted, tend to accumulate in the extraction instrument, ultimately interfering with its proper operation. Accordingly, frequent internal cleaning of the extraction instrument is required.

### SUMMARY OF THE INVENTION

This invention addresses the problem of sorting hair follicles by providing plural collectors, and a diverter apparatus for directing extracted follicular units to a selected one of the collectors. By operating the diverter apparatus, preferably by means of a set of foot pedals, the surgeon, while performing a series of follicle extractions, can direct each extracted follicle to an appropriate collector. For example, the follicle can be directed to an appropriate one of three collectors depending on whether it comprises a single hair, two hairs, or three or more hairs. The surgeon can observe the type of follicle visually, using a magnifying television monitor, immediately prior to its extraction, and direct it to an appropriate collector by depressing an associated foot pedal.

The apparatus for extracting hair follicles for use in transplantation, in accordance with a first aspect of the invention, comprises a handpiece, a diverter unit, and a plurality of follicular unit collectors. The handpiece has a rotatable punch for extracting a follicular unit from a patient's scalp. The diverter unit has an inlet and plural outlets. A first channel connects the handpiece to the inlet of the diverter unit for transport of extracted follicular units from the rotatable punch to the diverter unit. Each of the follicular unit collectors is connected through one channel of a set of said second channels to an associated outlet of the diverter unit, so that, by selective valve operation, an extracted follicular unit can be directed to a selected one of the collectors.

More specifically, an apparatus for extracting hair follicles in accordance with a first aspect of the invention can comprise an instrument with a hollow rotating needle, a plurality of collectors, and a diverter. The instrument includes an instrument body having a straight, elongated, hollow needle protruding therefrom along an axis. The needle is rotatable about the axis, and has axially spaced distal and proximal openings communicating with each other through an internal passage within the needle extending in the direction of the axis. A motor is connected to the needle for rotating the needle about the axis. The plurality of collectors is provided for receiving hair follicles. The diverter has an inlet opening and a plurality of outlet openings. A first channel is connected from the proximal opening of the needle to the inlet opening of the diverter. Each of a plurality of second channels is connected from one of the outlet openings of the diverter to one of the collectors. Thus, the first channel, the internal channel of the diverter, and any one of the second channels connected to the first channel through the diverter constitute a passage for connecting the needle to one of the collectors. A pump is connectible to each such passage for establishing a flow of air through each passage in a direction from the needle toward the collector connected to the passage. The flow of air is sufficient to transport hair follicles from the distal opening of the needle to the collector.

The diverter can include a movable valve member having an internal channel connected to the inlet opening for selectably connecting the inlet opening to each of the outlet openings. Alternatively, the apparatus can include plural valves, each associated with one of the collectors, for selectably establishing flow of air from the diverter to any selected one of the collectors while preventing flow of air from the diverter to the other collectors.

In another aspect, the invention addresses the problem of sealing in a follicle extraction apparatus utilizing suction. A novel seal construction reduces the accumulation of blood, tissue and other materials in the mechanism of the extraction instrument. The apparatus in accordance with this aspect of the invention is similar to the above-described instrument in that it comprises an instrument body having a straight, elongated, hollow needle protruding therefrom along an axis. The needle is rotatable about the axis, and has axially spaced distal and proximal openings communicating with each other through an internal passage within the needle extending in the direction of the axis. A motor is connected to the needle for rotating the needle about the axis.

A passage is connected to the needle and to a collector for receiving hair follicles. A pump connected to the passage establishes a flow of air through the passage in a direction from the needle toward the collector, the flow of air being sufficient to transport hair follicles from the distal opening of the needle to the collector.

A cylindrical wall surrounds a proximal portion of the hollow needle and forms a peripheral boundary of a compartment. A closure forms a proximal boundary of the compartment and has an opening through which the passage extends. The passage communicates with the proximal opening of the hollow needle. A seal disposed in the compartment, is in contact with at least a part of the proximal portion of the needle and with the cylindrical wall.

The seal is preferably composed of a toroidal spring in compression in a carrier composed of PTFE or a similar low-friction polymer. The spring urges the carrier against the rotating needle and against the interior wall of the seal compartment, thereby ensuring a continuous vacuum drawn through the needle and avoiding contamination.

Another aspect of the invention relates to an apparatus for the extraction of hair follicles, in which the hair follicles are collected in a closed system and automatically irrigated periodically with a spray of cooled saline solution to ensure their viability during the interval between extraction and implantation.

The apparatus in accordance with this aspect of the invention comprises an instrument body having a straight, elongated, hollow needle protruding therefrom along an axis. The needle is rotatable about the axis, and has distal and proximal openings communicating with each other through an internal passage within the needle, extending in the direction of the axis. A motor is provided for rotating the needle about the axis. The apparatus also comprises a collector for receiving hair follicles, a passage connected to the needle and to the collector, and a pump connected to the passage for establishing a flow of air through the passage, in a direction from the needle toward said collector, and sufficient to transport hair follicles from the distal opening of the needle to the collector. The apparatus is characterized by a spray nozzle in the collector, arranged to direct a spray of saline solution toward hair follicles received in the collector.

The collector preferably comprises a canister and an internal basket within the canister, the basket having a supporting surface for supporting hair follicles transported from the distal opening of the needle to the collector. The basket can be composed of a material sufficiently flexible that the basket can be turned inside-out when removed from the canister in order to facilitate removal of collected hair follicles from the basket.

The collector is an enclosure preferably having a first port, connected to the passage leading from the needle, through which hair follicles can enter the enclosure, a second port through which a supply of saline solution can flow to the spray nozzle, and a third port connected to the pump. The first, second and third ports are the only openings connecting the interior of the collector to the exterior thereof. The pump is thereby connected to the first port through the collector so that the pump draws extracted hair follicles through the passage to the collector.

The apparatus can include a container for holding a supply of saline solution, and a first conduit connected to the container and to the spray nozzle for delivery of saline solution from the container to the spray nozzle for spraying onto follicles received in the collector. The pump is connected to the interior of the collector through a second conduit preferably having an opening within the collector at a level below the level of the follicle supporting surface of the basket.

A valve provided in the first conduit can be opened periodically by a control to allow saline solution to flow through the first conduit to the spray nozzle and to be sprayed toward hair follicles on the supporting surface of the basket as a result of a difference between the pressure in the saline supply container and a lower pressure within the collector resulting from the action of the pump.

The second conduit preferably includes a reservoir having sufficient volume to maintain the pressure in the collector canister at a level lower than the pressure in the saline supply container while the valve in the first conduit is opened by the control. The reservoir also reduces pressure variations in the system.

A cooling unit may be arranged to maintain the temperature of the supply of saline solution in the saline container at a temperature below ambient temperature. The cooling unit can also cool saline solution remaining in the bottom of the collector.

### BRIEF DESCRIPTION OF THE DRAWINGS

FIG. 1 is a perspective view of a hair follicle extraction handpiece in accordance with the invention;

FIG. 2(a) is a cross-sectional view of a part of the handpiece of FIG. 1, showing internal details, including a chuck for holding the needle and a seal ring, and also showing a portion of a channel through which extracted follicular units travel;

FIG. 2(b) is an enlarged cross-sectional view of a portion of FIG. 2(a), showing the chuck and the seal ring.

FIG. 3 is a vertical cross-sectional view of the interior of a diverter valve;

FIG. 4 is a horizontal cross-sectional view of the interior of the diverter valve;

FIG. 5 is a schematic diagram of an apparatus in which plural valves, each associated with a different collector, are provided for directing extracted follicular units to selected collectors;

FIG. 6 is a perspective view of a console associated with the handpiece, including plural follicle-collecting filters and foot pedals for operating the diverter valve;

FIG. 7 is a cross-sectional view of a follicle-collecting filter;

FIG. 8 is a perspective view of an alternative console having liquid-containing follicle collectors;

FIG. 9 is a cross-sectional view of a liquid-containing follicle collector; and

FIG. 10 is a schematic cross-sectional view of another alternative console, having a follicle collector, a vacuum reservoir, a liquid reservoir for saline solution, and a cooling unit.

### DETAILED DESCRIPTION OF THE PREFERRED EMBODIMENTS

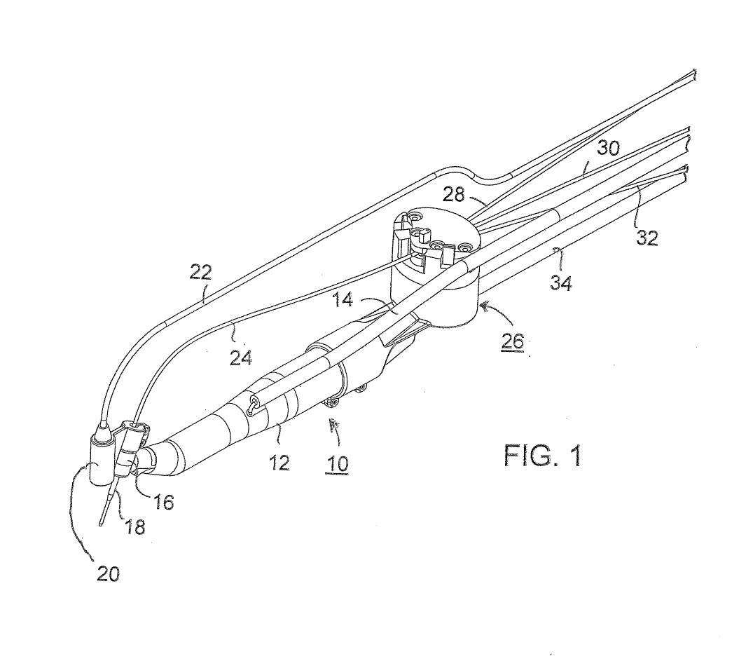

FIG. 1 shows a handpiece 10 for hair follicle extraction. The handpiece is composed of an elongated instrument body having a handle portion 12 with a shape and size such that it can be gripped manually by a surgeon and moved easily from one location to another on a patient's scalp. A motor (not shown) is provided inside the handle. The motor in the handpiece shown is an electric motor powered through a flexible cable 34. However, other kinds of motors, such as air-operated motors, can be used.

An instrument head 16 at one end of the handle portion 12 is shaped generally in the form of a cylinder, having an axis disposed in oblique relation to the long dimension of the handle. A hollow needle 18 protrudes from the cylindrical instrument head 16 along the axis in a direction such that the needle is disposed at an obtuse angle in relation to the handle. The needle is rotatable by the motor through gearing inside the head 16, and the motor can be turned on and off by a control (not shown), which can be either a foot-operated control or a manual control on the handle portion of the handpiece. Optionally, provisions can be made for operator control of the speed of operation of the motor.

Attached to the handpiece adjacent head 16 is a housing 20 for an electronic camera, e.g., a CCD camera, for generating an image of the area of the patient's scalp being operated upon. The image can be displayed in enlarged form on a video screen to assist the surgeon in extracting hair follicles. The camera can be connected to a video display apparatus through cable 22.

A tube 24 extends from the head 16 for carrying hair follicles extracted by the needle to a diverter assembly 26 mounted on the handpiece adjacent the proximal end of the handle. The diverter assembly is provided with three flexible exit cables, 28, 30 and 32, for carrying an extracted hair follicle to a selected one of three follicle collectors (not shown in FIG. 1). If the diverter includes a movable member, the diverter is powered and controlled through a flexible cable (not shown). Conduit 14 is an irrigation conduit.

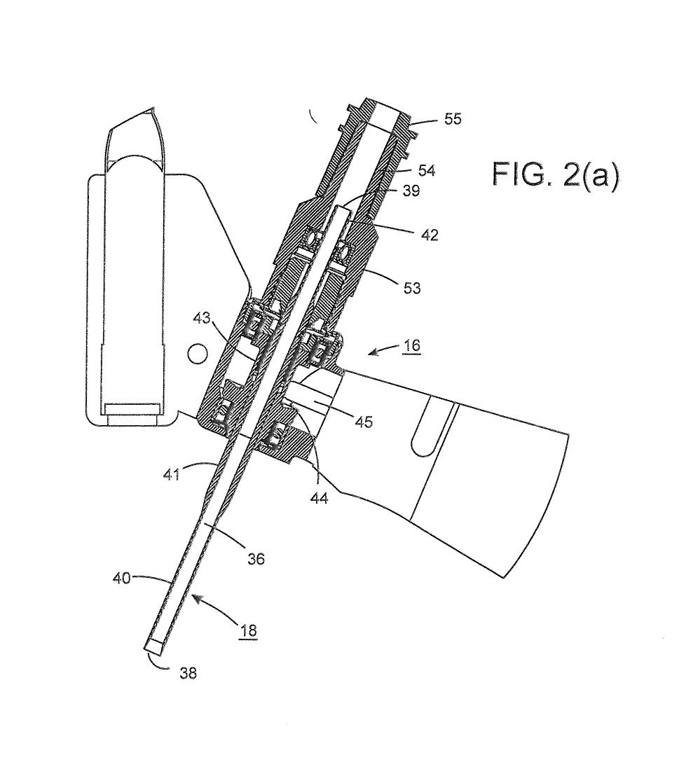

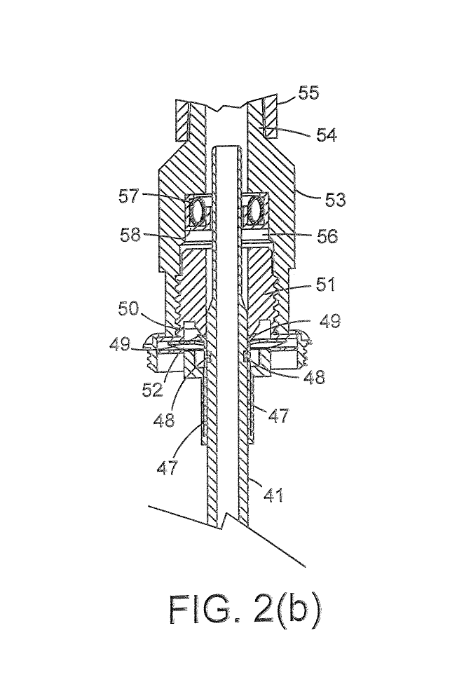

As shown in FIG. 2(a), needle 18 is a hollow needle having a smooth internal passage 36 extending from its distal end 38 to its proximal end 39. A part 40 of the needle extending proximally from its distal end 38 has a thin wall, while an intermediate part 41 of the needle has a thick wall. The distal end of the needle has an internal bevel forming a sharp circular cutting edge having a diameter equal to the external diameter of the distal part 40. A part 42 of the needle, extending proximally from the intermediate part 41, is narrower than the intermediate part.

The needle 18 extends through a sleeve 43, which is mounted for rotation in bearings inside the head 16. The sleeve is provided with beveled gear teeth which mesh with teeth on a gear 44 rotated by shaft 45 of the motor (not shown) inside the handle.

As shown in FIG. 2(b), the needle extends through a chuck 46, which is connected to, and driven by, the sleeve 43 (FIG. 2(a)). The chuck includes plural spring elements 47, each of which has a projection 48 that extends into an external groove on intermediate part 41 of the needle. The groove extends part way around the needle. The projections 48, by engaging the groove, prevent the needle from moving axially in the sleeve. At the same time, the projections engage end walls of the groove to ensure a positive connection between the sleeve 44 and the needle and to ensure that the needle rotates with the sleeve. The spring members have flared ears 49, which are engageable with the beveled end 50 of a threaded cap 51. A spring washer 52 is in compression between the cap and the chuck. The cap is threaded into a fitting 53 which has at its proximal end a connector 54, which mates with a connector 55 on tube 24 (FIG. 1). The connectors 54 and 55 can be any of a variety of fluid-tight connectors including tapered connectors and various forms of interlocking “Luer” connectors.

Disposed within a seal compartment 56 formed inside fitting 53, is a seal, composed of a toroidal spring 57 in compression inside a receiver 58, which is preferably formed of poly (tetrafluoroethylene) PTFE, or a similar low-friction polymer. The seal allows rotation of the needle while ensuring that the vacuum drawn through line 24 (FIG. 1) is maintained, and prevents contamination of the needle drive mechanism by blood, tissue and other materials drawn through the needle.

The needle can be removed from the chuck readily by pressing fitting 53 in the axial direction against spring 52. The beveled end 50 of cap 51 presses ears 49 outward, releasing the engagement of projections 48 with the groove in the intermediate part of the needle.

Alternative arrangements for holding the needle in the chuck can be used. For example, splines can be provided on the needle and inside the sleeve for positive mutual engagement and various other forms of detents can be used to prevent axial movement of the needle relative to the sleeve.

Other forms of seals can also be used. For example, the seal can be composed of one or more resilient O-rings made of synthetic rubber or a similar material disposed in compartment 56 and surrounding and in contact with a part of the proximal portion of the needle and in contact with the cylindrical inner wall of compartment 56. Axial compression of the O-rings can cause them to press radially against the needle and the cylindrical wall of the compartment.

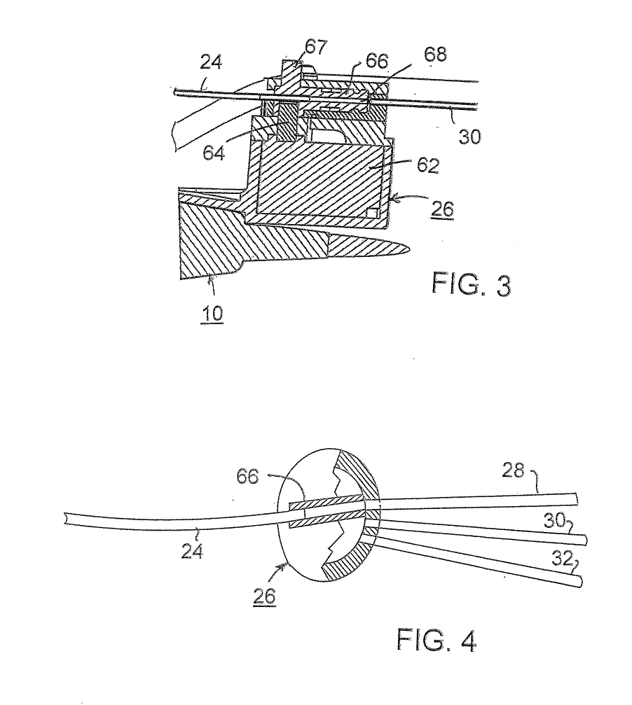

As shown in FIGS. 3 and 4, the diverter assembly 26 comprise a stepping motor 62 having a shaft 64 engaged with a movable valve element 66. The valve element has a mounting shaft 67, which is coaxial with stepping motor shaft 64. The valve element, and which allows the valve element to rotate through a range of angles.

The valve element 66 has an internal through passage 68, which receives an end of tube 24. Tube 24 should have sufficient flexibility to allow the valve element to be rotated about the axis of shafts 64 and 66 by the motor 62 through a limited angular range.

The motor is preferably a stepping motor capable of moving the valve element accurately to any selected one of three positions, in which the passage 68 of the valve element is in register with an end opening of one of the three exit tubes 28, 30 and 32, respectively (FIG. 1). Thus, the diverter enables the surgeon to select any one of the three exit tubes for delivery of an extracted hair follicle. By observing the hair follicle before extraction by the use of the camera on the handpiece and an associated video screen, the surgeon can determine the type of hair follicle (one hair, two hairs or three or more hairs) and route it to an appropriate collector, thereby obviating a subsequent, time-consuming, sorting operation. It is also possible to classify hair follicles by optical scanning, and to operate the diverter automatically in response to the optical scanning apparatus.

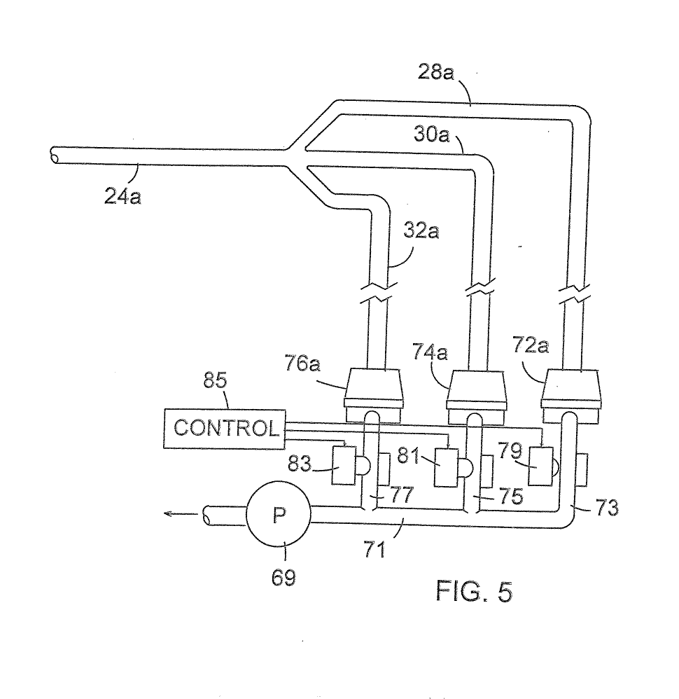

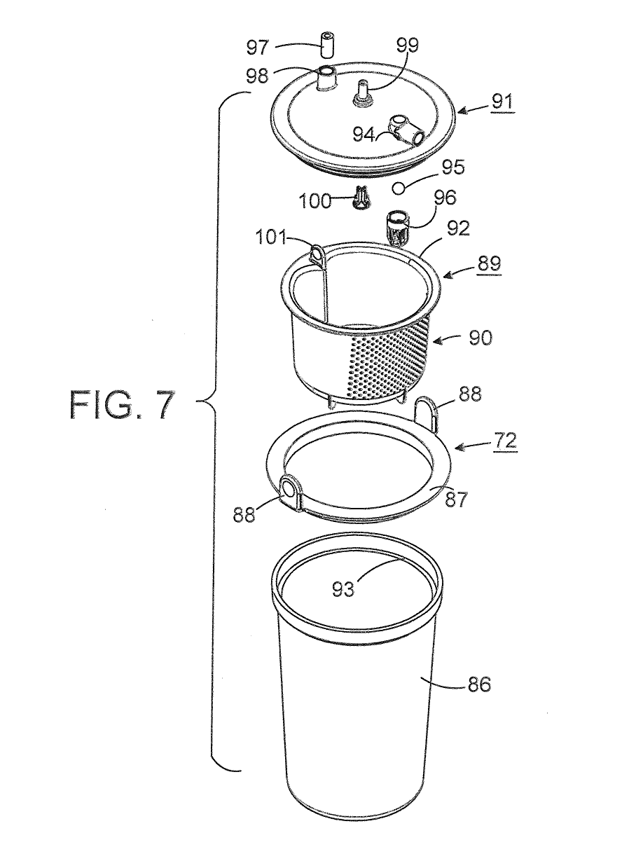

In the alternative apparatus depicted schematically in FIG. 5, the diverter 26a is in the form of a branched connection having an inlet and three outlets. The inlet is connected to the first channel 24a, which corresponds to tube 24 in FIG. 1. The outlets are connected respectively to exit tubes 28a, 30a and 32a, which correspond to exit tubes 28, 30 and 32 in FIG. 1. Each of three follicular unit collectors 72a, 74a and 76a, may have a construction similar or identical to that of collector 72 in FIG. 7. The follicular units are connected to the respective exit tubes 28a, 30a and 32a, and are connected to a suction pump 69 though line 71 and branch lines 73, 75 and 77. The branch lines are provided with pinch valves 79, 81 and 83, respectively, which are electrically operated pinch valves responsive to a control unit 85. The control unit may be operated by foot pedals or by any of various manual or automatic controls. The control should open only one of the three pinch valves at any given time, maintaining the other two valves in a closed condition. Thus valves 81 and 83 are closed while valve 79 is shown in an opened condition.

In operation of the apparatus of FIG. 5, an extracted follicular unit follows a path from the needle, through tube 24a and through the diverter 26a to the collector associated with the open pinch valve, in this case collector 72a. A next follicular unit can be directed to a different collector.

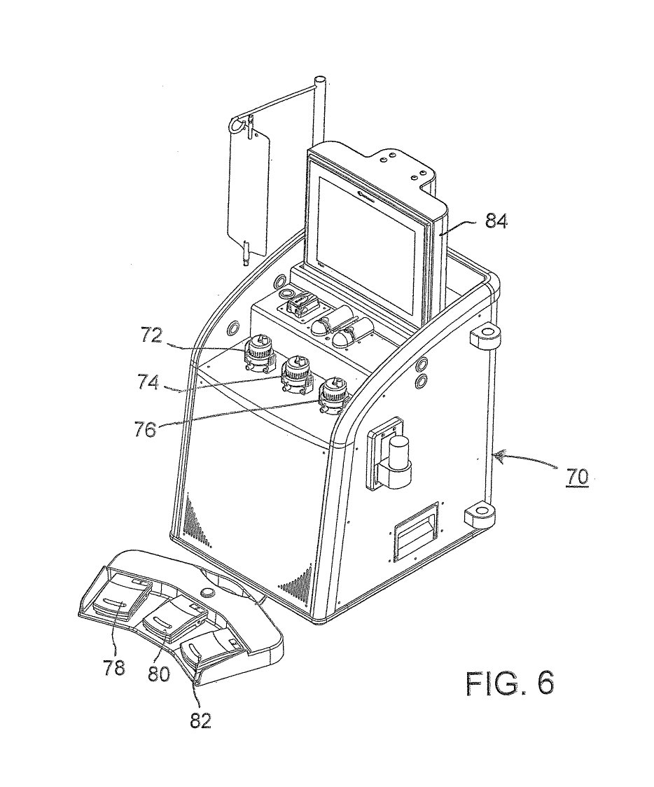

FIG. 6 shows a console 70 for use with the handpiece. the tubes and cables extending from the handpiece as shown in FIG. 1 can be connected to the console, which can then be used to control the motor in the handpiece, to operate the diverter for selection of a path for delivery of each extracted follicle, to display the image of the transmitted by the camera on the handpiece, to collect and preserve the extracted hair follicles in each of plural collectors, and to count the hair follicles delivered to each collector by the provision of a counting device (not shown).

Console 70 includes three collectors 72, 74 and 76, which are connected respectively to diverter exit cables, 28, 30 and 32. Foot pedals 78, 80 and 82, which are connected to the console, are used to control the stepper motor in the diverter for selection of a collector from collectors 72, 74 and 76, or to control the pinch valve controller 85 in FIG. 5. The console is also provided with a touch screen 84 for controlling various aspects of operation of the apparatus, such as needle rotation speed, pump operation, suction, automatic delivery of saline spray to the follicular unit collectors, spray volume, spray interval, resetting a follicle counter, etc.

The collectors can be identical, and collector 72 is shown in FIG. 7. The collector comprises a cup-shaped outer canister 86, a ring-shaped seal 87 having a pair of grip tabs 88, a follicle collection basket 89 having perforations 90 both in its side walls and in its bottom, and a cover 91. When the collector is assembled, a rim 92 of the collection basket rests on a ledge 93 on the inner wall of the canister, the seal is inserted into the opening of the canister and the cover 91 is placed on top of the seal so that the outer part of the seal is disposed between the cover and the upper edge of the canister.

A vacuum line (not shown in FIG. 7) is connected to fitting 94 on the cover, and a check valve assembly, comprising a check ball 95 and a check ball retainer 96, limits the vacuum inside the collector. Follicular units extracted by the apparatus shown in FIG. 2 are delivered to the collector 72 through a flexible conduit, e.g. 28, 30 or 32 in FIG. 1 or 28a, 30a or 32a in FIG. 5, and through a fitting 97, which connects removably to port 98 in cover 91. Follicular units collected in basket 89 can be irrigated by saline solution introduced through port 99 in the cover and sprayed onto the follicular units by a spray nozzle 100.

The collection basket 89 has a grip tab 101, and can be made of a flexible material, such as silicone rubber, that allows it to be turned inside-out when removed from the canister in order to facilitate removal of collected follicular units.

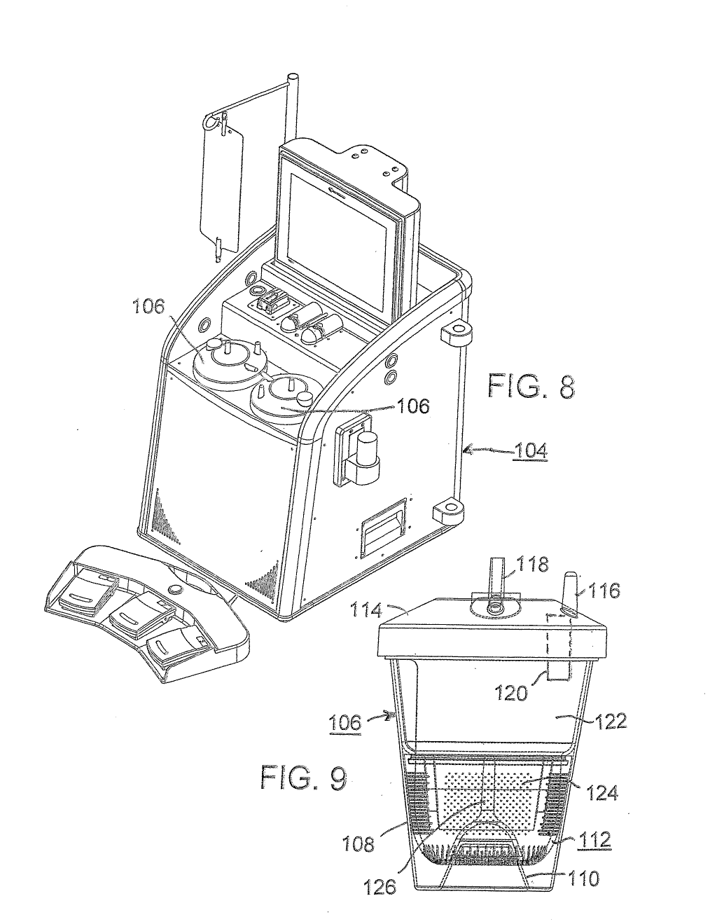

Console 104, shown in FIG. 8, is similar to console 70, except that it has two collectors 106, and they are designed to maintain the collected hair follicles immersed in a liquid as they accumulate.

As shown in FIG. 9, the collector 106 comprises a cup-shaped container 108 having an internal pedestal 110 on which rests an internal filter 112. The internal filter has plural sets of perforations, each perforation being of a size that an extracted hair follicle cannot pass through it. The container 108 is closed by a lid 114, which has a port 116 for connection to a follicular unit conducting tube and a centrally located port 118 for connection to a suction pump (not shown). The filter 112 is immersed in a saline solution within the container 108, so that the collected hair follicles remain immersed until they are harvested for implantation.

A porous element (not shown) is provided in the collector immediately below the suction port 18 to prevent rapid concentrated flow of air through the suction port, which could draw hair follicles into the suction port. The porous element can also expand when wet, so that its pores close, preventing water from being drawn through the suction port in the event that the collector is overfilled.

A baffle 120 is provided within the collector between the follicle-receiving port 116 and the suction port 118 to ensure that the hair follicles entering the collector drop into the liquid in the lower part of the container.

The internal filter 112 is composed of two separable parts: a rigid, frusto-conical upper part 122 and a flexible cup-shaped lower part 124, preferably composed of silicone rubber or a material having similar physical properties. The lower part grips the upper part and can be pulled away from the upper part. When the desired number of follicular units has been collected, the lid 114 is removed, the upper and lower internal parts 122 and 124 are removed as a unit from the outer container 108, and separated from each other. The lower part 124 is provided with an upright central post which can be grasped manually, enabling the lower part to be turned inside-out, so that collected hair follicles can be readily and gently dropped onto a flat sheet from which they can be retrieved individually for reimplantation.

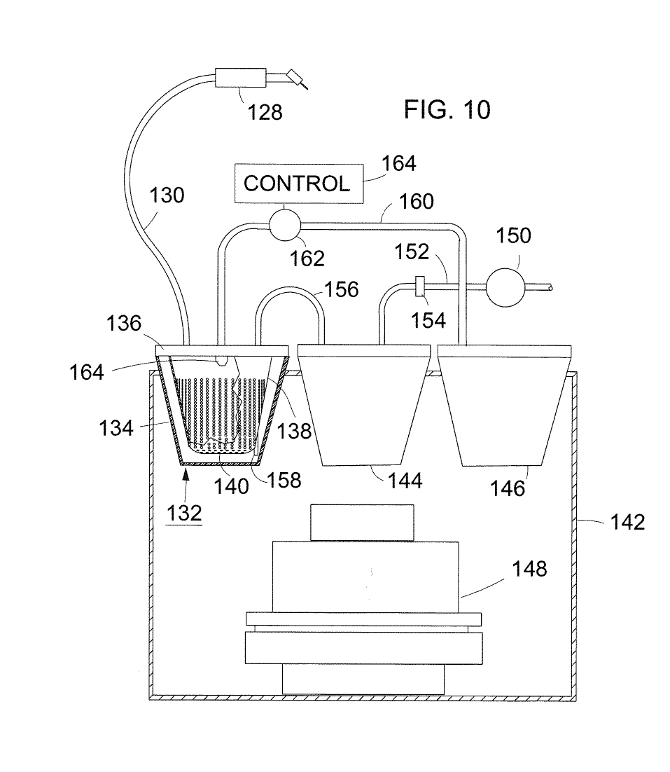

In the embodiment depicted in FIG. 10, handpiece 128 can be similar to the handpiece depicted in FIG. 1, but in this case the handpiece does not include a diverter assembly, and has only one flexible exit cable 130 for the transport of extracted hair follicles to a collector 132. The collector comprises a canister 134 having a removable lid 136. The collector can be similar to the collector depicted in FIG. 7. A basket 138, for collecting hair follicles transported through cable 130, is supported inside the canister 134. The basket is formed with an array of small perforations that allow the passage of air and liquid, but retain collected hair follicles. The perforations include perforations in the bottom 140 of the basket, but can include some perforations in the side of the basket. Perforations in the side of the basket need not completely surround the basket, and as in the case of the basket in the collector of FIG. 7, they can be provided on one side of basket. These perforations in the side wall of the basket may be provided to allow for sufficient flow of air to draw hair follicles into the basket through cable 130.

The collector canister 134 is supported in the top of an enclosed console 142 along with two similarly shaped canisters 144 and 146. Canister 144 serves as a vacuum reservoir, and canister 146 is a supply container for saline solution, which is automatically sprayed at intervals onto the hair follicles collected in the basket 138 of collector 132.

Also enclosed in the console 142 is a cooling unit 148. The cooling unit maintains the air inside the console at a temperature below ambient, preferably about 5° C. A Peltier effect thermoelectric air cooler is suitable for this purpose, and an example of a suitable air cooler is described in U.S. Pat. No. 6,530,231, granted Mar. 11, 2003 and U.S. Pat. No. 6,662,571, granted Dec. 16, 2003. The disclosures of both patents are here incorporated by reference.

Heat from the saline solution in canister 146 is transferred through the wall of the canister to the air inside the console, and the saline solution is thereby cooled. The cooling unit also cools saline solution remaining at the bottom of canister 134 and the air inside canister 134.

As shown in FIG. 10, a pump 150 draws a vacuum on the interior of the vacuum reservoir canister 144 through conduit section 152, which is provided with a filter 154. The interior of the vacuum reservoir is, in turn, connected, through a conduit section 156 to the interior of canister 134. The opening 158 of conduit section 156 is preferably located inside canister 134 at a level slightly below the level of the follicle supporting surface on the inside of the bottom 140 of basket 138. The basket 138 is shaped with an indented side wall (not shown) in order to provide room for conduit section 156 to extend past the basket to the lower part of the interior of canister 134.

A conduit 160, having a valve 162 is connected to transport saline solution from canister 146 to a spray nozzle 164 centrally located beneath the lid 136 of canister 134. The valve 162, which can be a pinch valve, is electrically controlled by a control unit 164, which automatically causes the valve to open at regular intervals during the operation of the apparatus, e.g., for a fraction of a second, every two or three minutes. When the valve 162 opens, the vacuum drawn by pump 150 causes saline solution to flow through conduit 160 to the nozzle 164 inside the canister so that the hair follicles collected at the bottom of the basket 138 are periodically irrigated by the saline solution spray.

The holes in the basket 138 prevent large quantities of liquid from collecting in the basket The holes in the bottom of the basket are preferably small enough that surface tension in the saline solution causes a small quantity of the solution to be retained in the bottom of the basket for continued irrigation of the hair follicles.

The vacuum reservoir canister 144 ensures that a relatively constant level of vacuum is continuously maintained in the interior of the collector canister 134 so that saline solution will be sprayed through nozzle 164 whenever valve 162 opens and that hair follicles will be drawn into the collector through flexible cable 130 whenever they are released to cable 130 from the handpiece. Thus pump 150, which can operate continuously, can be a relatively small capacity pump.

As will be apparent, the system in FIG. 10 is a closed system. The collected hair follicles are delivered from the handpiece through flexible cable 130 to the basket inside the the collector canister, and the only openings in the collector canister are the opening for cable 130, the opening for the saline solution conduit 160, and the opening for the vacuum connection through conduit section 156. The only openings in the vacuum reservoir canister 144, are the openings for the conduit sections 152 and 156. The saline solution supply canister is also closed except for the connection to conduit 160 and a small opening (not shown) in the lid of the canister 160 for the entry of air to displace the saline solution as it is exhausted from the supply canister through conduit 160.

What is claimed is:

1. Apparatus for extracting hair follicles for use in transplantation comprising:
an instrument body having a straight, elongated, hollow needle protruding therefrom along an axis, said needle being rotatable about said axis, and having axially spaced distal and proximal openings communicating with each other through an internal passage within the needle extending in the direction of said axis;
a motor connected to said needle for rotating said needle about said axis;
a plurality of collectors for receiving hair follicles;
a diverter having an inlet opening and a plurality of outlet openings;
a first channel connected from the proximal opening of the needle to the inlet opening of the diverter valve;
a plurality of second channels, each second channel of said plurality of channels being connected from one of said outlet openings of the diverter to one of said collectors, whereby said first channel and any one of said second channels connected to said first channel through the diverter constitute a passage for connecting said needle to one of said collectors; and
a pump connectible to each said passage for establishing a flow of air through each passage in a direction from said needle toward the collector connected to each passage, said flow of air being sufficient to transport hair follicles from the distal opening of the needle to the collector;
wherein said diverter includes a movable valve member having an internal channel for selectably connecting said inlet opening to any selected one of said outlet openings.

2. Apparatus according to claim 1, including a motor connected to said movable member of the diverter, for selectably connecting said first channel, through said internal channel of the movable valve member, to a selected one of said second channels.

3. Apparatus according to claim 2, including a set of foot pedals, for operating said motor, each of said foot pedals being operable to cause the motor to move the movable member of the diverter for connection of the first channel to a different one of said second channels.

4. Apparatus according to claim 3, wherein said motor is a stepping motor.

5. Apparatus for extracting hair follicles for use in transplantation comprising:
an instrument body having a straight, elongated, hollow needle protruding therefrom along an axis, said needle being rotatable about said axis, and having distal and proximal openings communicating with each other through an internal passage within the needle, extending in the direction of said axis;
a motor for rotating said needle about said axis;
a collector for receiving hair follicles;
a passage connected to said needle and to said collector;
a pump connected to said passage for establishing a flow of air through the passage in a direction from said needle toward said collector, said flow of air being sufficient to transport hair follicles from the distal opening of the needle to the collector;
a cylindrical wall surrounding a proximal portion of the hollow needle and forming a peripheral boundary of a compartment;
a closure forming a proximal boundary of said compartment and having an opening through which said passage extends, said passage communicating with the proximal opening of the hollow needle; and
a seal disposed in said compartment, said seal being in contact with at least a part of said proximal portion of the needle and with said cylindrical wall.

6. Apparatus according to claim 5, in which said seal is composed of an annular spring receiver having an inner wall in contact with said proximal portion of the hollow needle and an outer wall in contact with said cylindrical wall surrounding a proximal portion of the hollow needle, and a toroidal spring in compression between said inner and outer walls of the annular spring receiver, said toroidal spring urging said inner wall of the spring receiver against said proximal portion of the hollow needle and urging said outer wall of the spring receiver against said cylindrical wall surrounding said proximal portion of the hollow needle.

7. Apparatus according to claim 5, in which said seal is composed of an annular spring receiver composed of a low-friction polymer, said receiver having an inner wall in contact with said proximal portion of the hollow needle and an outer wall in contact with said cylindrical wall surrounding a proximal portion of the hollow needle, and a toroidal spring in compression between said inner and outer walls of the annular spring receiver, said toroidal spring urging said inner wall of the spring receiver against said proximal portion of the hollow needle and urging said outer wall of the spring receiver against said cylindrical wall surrounding said proximal portion of the hollow needle.

8. Apparatus according to claim 5, in which said seal is composed of an annular spring receiver composed of poly (tetrafluoroethylene), said receiver having an inner wall in contact with said proximal portion of the hollow needle and an outer wall in contact with said cylindrical wall surrounding a proximal portion of the hollow needle, and a toroidal spring in compression between said inner and outer walls of the annular spring receiver, said toroidal spring urging said inner wall of the spring receiver against said proximal portion of the hollow needle and urging said outer wall of the spring receiver against said cylindrical wall surrounding said proximal portion of the hollow needle.

9. Apparatus for extracting hair follicles for use in transplantation comprising:
an instrument body having a straight, elongated, hollow needle protruding therefrom along an axis, said needle being rotatable about said axis, and having distal and proximal openings communicating with each other through an internal passage within the needle, extending in the direction of said axis;
a motor for rotating said needle about said axis;
a collector for receiving hair follicles;
a passage connected to said needle and to said collector;
a pump connected to said passage for establishing a flow of air through the passage in a direction from said needle toward said collector, said flow of air being sufficient to transport hair follicles from the distal opening of the needle to the collector; and
a spray nozzle in said collector, the spray nozzle being arranged to direct a spray of saline solution toward hair follicles received in said collector.

10. Apparatus according to claim 9 in which said collector comprises a canister and an internal basket within said canister, said basket having a supporting surface for supporting hair follicles transported from the distal opening of the needle to the collector, and in which said spray nozzle is arranged to direct said spray of saline solution toward said supporting surface of the basket.

11. Apparatus according to claim 9 in which said collector comprises a canister and an internal basket within said canister, said basket having a supporting surface for supporting hair transported from the distal opening of the needle to the collector, and said basket being composed of a material sufficiently flexible that said basket can be turned inside-out when removed from the canister in order to facilitate removal of collected hair follicles from said basket.

12. Apparatus according to claim 9, in which said collector is an enclosure having a first port through which hair follicles can enter said enclosure, said first port being connected to said passage, a second port connected to said spray nozzle and through which a supply of saline solution can flow to said spray nozzle, and a third port connected to said pump, said pump being connected to said first port through said collector and in which said first, second and third ports are the only openings connecting the interior of said collector to the exterior thereof.

13. Apparatus according to claim 9, in which said pump is connected, through said collector to said passage.

14. Apparatus according to claim 9 including means for effecting automatic delivery of said spray of saline solution toward hair follicles received in said collector.

15. Apparatus according to claim 9, in which said collector comprises a canister and an internal basket within said canister, said basket having a supporting surface for supporting hair transported from the distal opening of the needle to said internal basket, said apparatus including a container for holding a supply of saline solution, a spray nozzle within said canister for spraying saline solution toward said supporting surface of the basket, and a conduit connected to said container and to said nozzle for delivery of saline solution from said container to said nozzle for spraying onto follicles received in said collector.

16. Apparatus according to claim 9, in which said collector comprises a canister and an internal basket within said canister, said basket having a supporting surface for supporting hair transported from the distal opening of the needle to said internal basket, said apparatus including a container for holding a supply of saline solution, a spray nozzle within said canister for spraying saline solution toward said supporting surface of the basket, and a first conduit connected to said container and to said nozzle for delivery of saline solution from said container to said nozzle for spraying onto follicles received in said collector, and in which said pump is connected to the interior of said collector through a second conduit having an opening within said collector at a level below the level of said supporting surface of the basket.

17. Apparatus according to claim 9, in which said collector comprises a canister and an internal basket within said canister, said basket having a supporting surface for supporting hair transported from the distal opening of the needle to said internal basket, said apparatus including a container for holding a supply of saline solution, a spray nozzle within said canister for spraying saline solution toward said supporting surface of the basket, a first conduit connected to said container and to said nozzle for delivery of saline solution from said container to said nozzle for spraying of said saline solution onto hair follicles received in said collector, in which said pump is connected to the interior of said collector through a second conduit having an opening within said collector at a level below the level of said supporting surface of the basket, a valve in said first conduit, and a control for periodically opening said valve to allow saline solution to flow through said first conduit to said spray nozzle and to be sprayed toward hair follicles on said supporting surface of the basket as a result of a difference between the pressure in said container for holding a supply of saline solution and a lower pressure within said collector resulting from the action of said pump.

18. Apparatus according to claim 9, in which said collector comprises a canister and an internal basket within said canister, said basket having a supporting surface for supporting hair transported from the distal opening of the needle to said internal basket, said apparatus including a container for holding a supply of saline solution, a spray nozzle within said canister for spraying saline solution toward said supporting surface of the basket, a first conduit connected to said container and to said nozzle for delivery of saline solution from said container to said nozzle for spraying of said saline solution onto follicles received in said collector, in which said pump is connected to the interior of said collector through a second conduit having an opening within said collector at a level below the level of said supporting surface of the basket, a valve in said first conduit, a control for periodically opening said valve to allow saline solution to flow through said first conduit to said spray nozzle and to be sprayed toward hair follicles on said supporting surface of the basket as a result of a difference between the pressure in said container for holding a supply of saline solution and a lower pressure within said collector resulting from the action of said pump, and a reservoir in said second conduit between said pump and said opening of the second conduit, said reservoir having sufficient volume to maintain the pressure in said canister at a level lower than the pressure in said container for holding a supply of saline solution while said valve is opened by said control.

19. Apparatus according to claim 9, in which said collector comprises a canister and an internal basket within said canister, said basket having a supporting surface for supporting hair transported from the distal opening of the needle to said internal basket, said apparatus including a container for holding a supply of saline solution, a spray nozzle within said canister for spraying saline solution toward said supporting surface of the basket, a conduit connected to said container and to said nozzle for delivery of saline solution from said container to said nozzle for spraying onto follicles received in said collector, and a cooling unit arranged to maintain the temperature of the supply of saline solution in said container at a temperature below ambient temperature.

20. Apparatus according to claim 9, in which said collector comprises a canister and an internal basket within said canister, said basket having a supporting surface for supporting hair transported from the distal opening of the needle to said internal basket, said apparatus including a container for holding a supply of saline solution, a spray nozzle within said canister for spraying saline solution toward said supporting surface of the basket, a first conduit connected to said container and to said nozzle for delivery of saline solution from said container to said nozzle for spraying of said saline solution onto hair follicles received in said collector, in which said pump is connected to the interior of said collector through a second conduit having an opening within said collector at a level below the level of said supporting surface of the basket, a valve in said first conduit, a control for periodically opening said valve to allow saline solution to flow through said first conduit to said spray nozzle and to be sprayed toward hair follicles on said supporting surface of the basket as a result of a difference between the pressure in said container for holding a supply of saline solution and a lower pressure within said collector resulting from the action of said pump, and a cooling unit arranged to maintain the temperature of the supply of saline solution in said container at a temperature below ambient temperature.
